(19) United States
(12) Patent Application Publication
(10) Pub. No.: US 2005/0101864 A1
(43) Pub. Date: May 12, 2005

(54) Ultrasound diagnostic imaging system and method for 3D qualitative display of 2D border tracings

(76) Inventors: Chuan Zheng, Bedford, MA (US); Ivan S. Salgo, Andover, MA (US)

Correspondence Address: PHILIPS INTELLECTUAL PROPERTY & STANDARDS, P.O. BOX 3001, BRIARCLIFF MANOR, NY, 10510, US

(21) Appl. No.: 10/965,612

(22) Filed: Oct. 14, 2004

Related U.S. Application Data
(60) Provisional application No. 60/513,631, filed on Oct. 23, 2003.

(51) Int. Cl. A61B 8/00

(52) U.S. Cl. 600/443; 128/916

(57) ABSTRACT

A method and system for generating a three-dimensional (3D) qualitative display (10,110,144) in an ultrasound system (30) include generating a first and a second two-dimensional (2D) slice (102,106,108) from a 3D data set of a 3D volume view of an ultrasound image. The first and second 2D slices (102,106,108) define a first and second plane of the 3D volume view along a first axis, wherein the second plane is orthogonal to the first plane. First and second border tracings (122,124) are generated around a portion of interest in the first and second 2D slices (102,106), respectively. A display (48) then displays (10,110) representations of the first and second border tracings within a single 3D view (10,128,130,146), wherein the 3D view provides an indication of alignment distortion of the first and second border tracings along the first axis. In one embodiment, at least one additional 2D slice defines an additional plane of the 3D volume view along a second axis, orthogonal to the first and second planes. Furthermore, at least one additional border tracing generated. The display (48) then further displays the at least one additional border tracing along the second axis within the single 3D view for providing an indication of alignment distortion of the first, second, and at least one additional border tracing along the first and second axes.

### CROSS REFERENCE TO RELATED CASES

Applicants claim the benefit of Provisional Application Ser. No. 60/513,631, filed Oct. 23, 2003.

### BACKGROUND OF THE INVENTION

The present disclosure generally relates to medical ultrasound imaging, and, more particularly, to an ultrasound diagnostic imaging system and method for 3D qualitative display of manual 2D LV border tracings.

Echocardiographic ultrasonic imaging systems are used to assess the performance of the heart. Cardiac performance can be assessed qualitatively with these systems, such as by observing the blood flow through vessels and valves and the operation of heart valves. Quantitative measures of cardiac performance can also be obtained with such systems. For instance, the velocity of blood flow and the sizes of organs and cavities such as a heart chamber can be measured. These measures can produce quantified values of cardiac performance such as ejection fraction and cardiac output.

One example of a method and apparatus for measuring the volume of a heart chamber is described in U.S. Pat. No. 5,322,067 (Prater et al.). In the method of the Prater et al. patent, a clinician acquires a sequence of ultrasound images of a cavity to be measured, for example, the left ventricle of the heart. The clinician freezes one of the images on a display screen and traces a fixed region of interest (ROI) around the cavity of the heart chamber. The defined ROI should be large enough to encompass the heart chamber when the heart is fully expanded.

### SUMMARY OF THE INVENTION

The ultrasound system then processes the pixels in the ROI in each image in the sequence to determine those pixels that are blood pixels in the left ventricle. The left ventricle is then segmented into strips and the area of the strips is calculated. Each strip is then conceptually rotated about its center to define a disk and the volume of each disk is calculated. By summing the volumes of the disks in each image, the volume of the heart chamber can be determined at each point in the heart cycle for which an image was acquired. The calculated volumes can then be displayed numerically as a function of time, or a waveform representative of left ventricle volume as a function of time can be produced, thereby showing the clinician the changes in left ventricular volume over the heart cycle.

The method of the Prater et al. patent uses manual input from the clinician to define a ROI by a manual tracing. Accordingly, the method is performed on a stored image loop due to the need for manual input. In addition, the method of disks (Simpson's rule) volume estimation assumes that each disk is uniformly circular, which may not be the case. It would be desirable to estimate cavity volumes that are more closely related to the true shape of the anatomy rather than having to rely on an assumption of geometric uniformity of the anatomy, thus producing more accurate volume measures.

Three-dimensional ultrasound imaging systems generally include an ultrasound probe to direct ultrasound waves to, as well as to receive reflected ultrasound waves from, a target volume of a subject under examination. The ultrasound probe is swept over the target volume and the reflected ultrasound waves are conveyed to a computer. Using the computer, successive two-dimensional images of the target volume are reconstructed to form a three dimensional image of the target volume. The three-dimensional image is displayed upon a display screen.

The displayed image can be manipulated by a user via a user interface. In one such system, the entire displayed image may be rotated about an arbitrary axis, a surface of the displayed image may be translated to provide different cross-sectional views of the image and a selected surface of the displayed image may be rotated about an arbitrary axis. The three-dimensional rendering of the target volume might also be manipulated using automated techniques, such as an automatic border tracing technique. For example, an automated border tracing technique might be performed by the ultrasound system as ultrasound images are acquired. However, such three-dimensional renderings of the target volume of interest may still contain inaccuracies, for example, misalignment of horizontal and vertical axes.

Accordingly, an improved ultrasound technique for overcoming the problems in the art is desired.

According to one embodiment, a method for generating a three-dimensional (3D) qualitative display in an ultrasound system includes generating a first two-dimensional (2D) slice from a 3D data set that is used to generate a 3D volume view of an ultrasound image. The first slice defines a first plane of the 3D volume view along a first axis. The method further includes generating a second 2D slice from the 3D data set of the 3D volume view. The second slice defines a second plane of the 3D volume view along the first axis, the second plane being orthogonal to the first plane. First and second border tracings are then generated around a portion of interest in the first and second 2D slices, respectively. In addition, representations of the first and second border tracings are displayed within a single 3D view, wherein the 3D view provides an indication of alignment distortion of the first and second border tracings along the first axis.

In another embodiment, at least one additional 2D slice of the 3D volume view is generated, the at least one additional slice defining at least one additional plane of the 3D volume view along a second axis, wherein the at least one additional plane is orthogonal to the first and second planes. At least one additional border tracing is generated around the portion of interest in the at least one additional 2D slice. The display provides for also displaying the at least one additional border tracing along the second axis, the 3D view providing an indication of alignment distortion along the first and second axes.

### BRIEF DESCRIPTION OF THE DRAWINGS

FIG. 1 is a three-dimensional (3D) display view of short axis border tracings and long axis border tracings of a target volume according to one embodiment of the present disclosure;

FIG. 2 is a block diagram view of an ultrasound diagnostic imaging system for implementing a three-dimensional (3D) qualitative display of 2D LV border tracings according to one embodiment of the present disclosure;

FIG. 3 is a flow diagram view of a method for generating a three-dimensional (3D) qualitative display of 2D LV border tracings in an ultrasound diagnostic imaging system according to one embodiment of the present disclosure;

FIG. 4 is an illustrative view of a 3D volume and portions thereof according to one embodiment of the present disclosure;

FIGS. 5, 6, 7, and 8 are illustrative views of a 3D volume and portions thereof according to an embodiment of the present disclosure;

FIG. 9 is an illustrative view of various 2D slices of a 3D volume according to one embodiment of the present disclosure; and

FIG. 10 is an illustrative view of a 3D volume and portions thereof, including a three-dimensional (3D) display view of short axis border tracings and long axis border tracings of a target volume according to one embodiment of the present disclosure.

### DESCRIPTION OF THE PREFERRED EMBODIMENTS

In connection with seeking improvements to ultrasound diagnostic imaging systems, the inventors of the embodiments of the present disclosure have discovered from 3D data sets used in constructing 3D volume views of an ultrasound image, in particular, for assessment of the left ventricle (LV) of the human heart, that short axis traces of the 3D volume views do not always coincide with long axis traces of the 3D volume views. The discrepancy between alignment of the short axis traces and the long axis traces is significant in that the traces should line up with each other.

According to an embodiment of the present disclosure, a method of implementing a 3D qualitative display includes the use of manual or automated 2D LV border tracings and the displaying of short axis traces and long axis traces of the 2D LV border tracings together. In other words, a display is provided that shows how a series of 2D borders drawn manually or automatically on multi-planar reformatted (MPR) views line up in 3D space. If the apex of the heart is selected incorrectly, i.e., by the incorrect MPR slice, then the error will show up as a misalignment of the borders. From the display of the short axis traces and long axis traces, the method provides for determining whether the 2D LV border tracings line up (or don't line up) in three dimensions. Upon obtaining an illustrative display indication of the alignment (or misalignment), appropriate adjustment(s) for aligning the short and long axis tracings can be made, thus providing an indication of an accuracy of a corresponding 3D volume view.

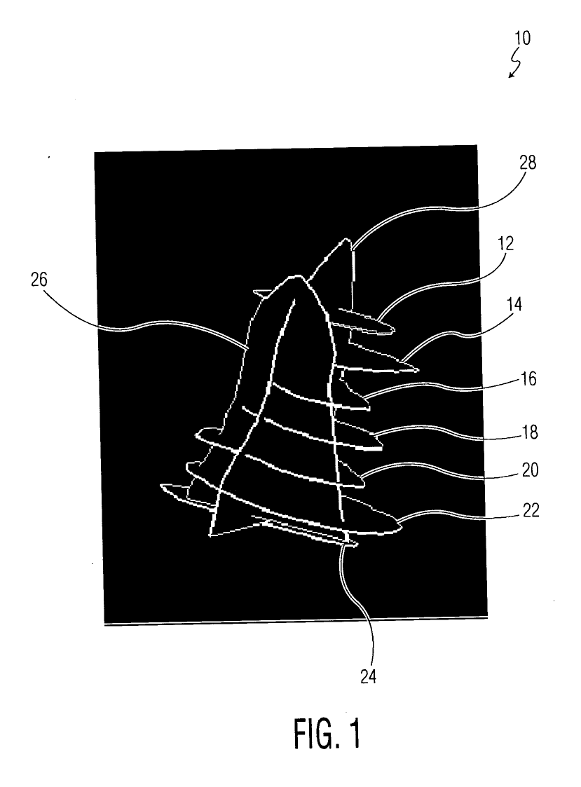

FIG. 1 is a three-dimensional (3D) display view of short axis border tracings and long axis border tracings of a target volume according to one embodiment of the present disclosure. More particularly, as illustrated in FIG. 1, the 3D display view 10 illustrates an example wherein the short axis borders (indicated by reference numerals 12, 14, 16, 18, 20, 22, and 24) do not line up with the long axis borders (indicated by reference numerals 26 and 28). Note also that the two long axis borders 26 and 28 do not line up either. Accordingly, the display view 10 of the short axis border tracings and long axis border tracings provides a useful tool for identifying and understanding the phenomena of the misalignment of the short axis and long axis border tracings. In addition, misalignment can be viewed in terms of an alignment distortion. The alignment distortion can include non-alignment of at least one border tracing along a first axis with at least one border tracing along a second axis, as will be discussed further herein. Display view 10 is also useful in connection with 3D segmentation and quantification. Border tracings as discussed herein can include any suitable method for manual border tracings and/or automatic border tracings, as is known in the art.

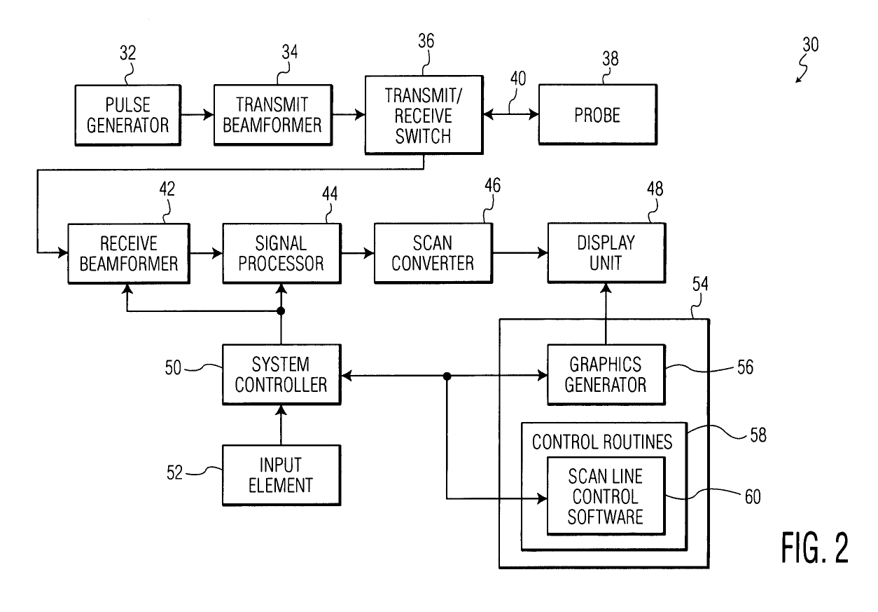

FIG. 2 is a block diagram view of an ultrasound diagnostic imaging system for implementing a three-dimensional (3D) qualitative display of 2D LV border tracings according to one embodiment of the present disclosure. Ultrasound diagnostic imaging system 30 includes a pulse generator 32, coupled to a transmit beamformer 34, coupled to a transmit/receive switch 36. An ultrasound probe 38 couples to transmit/receive switch 36 via a cable 40. The transmit/receive switch 36 of ultrasound diagnostic imaging system 30 couples to a receive beamformer 42, which is coupled to a signal processor 44, which is further coupled to a scan converter 46, and a display unit 48.

Ultrasound diagnostic imaging system 30 further includes a system controller 50, the system controller 50 being responsive, in part, to signals received from an input element 52 coupled to the system controller 50. Input element 52 enables system user input, such as manual operation of one or more portions of the method according to the various embodiments of the present disclosure. Input element 52 can include any suitable computer system input element, such as a keyboard, mouse, trackball, pointer device, or other suitable input device. System controller 50 is further coupled to receive beamformer 42 and signal processor 44 for providing signals, such as control and other signals, to the respective devices.

Still further, ultrasound diagnostic imaging system 30 includes a unit 54 containing graphics generator 56 and control routines 58. Control routines 58 include scan line control software 60. System controller 50 bi-directionally couples with graphics generator 56, as well as with control routines 58 and scan line control software 60, for carrying out the various functions according to the embodiments of the present disclosure. Graphics generator 56 couples to display unit 48 for providing appropriate signals for display, further as discussed herein with respect to the embodiments of the present disclosure. Operation of the basic components of an ultrasound diagnostic imaging system is known in the art and only briefly discussed herein.

With reference still to FIG. 2, ultrasound probe 32 can include, for example, a two dimensional array transducer and a micro-beamformer. The micro-beamformer contains circuitry which controls the signals applied to groups of elements (“patches”) of the array transducer and does some processing of the echo signals received by elements of each group. Micro-beamforming in the probe advantageously reduces the number of conductors in the cable 40 between the probe 38 and the remainder of the ultrasound system 30. Such a probe can include one as described in U.S. Pat. No. 5,997,479 to Savord et al. and/or in U.S. Pat. No. 6,436,048 to Pesque, incorporated herein by reference. The pulse generator 32, transmit beamformer 34, and transmit/receive switch 36 provide control signals to the microbeamformer of the probe 38, instructing the probe 38 as to the timing, frequency, direction and focusing of transmit beams.

The system controller 50 and receive beamformer 42 operate to control beamforming of received echo signals by probe 38. The echo signals are formed into beams by beamformer 42. The system controller 50 and signal processor 44 then operate to process the signals from beamformer 42. That is, the echo signals are processed by signal processor 44 which performs digital filtering, B mode detection, and/or Doppler processing, and can also perform other signal processing such as harmonic separation, speckle reduction through frequency compounding, and other desired image processing. Signal processor 44 output processed signals to scan converter 46, wherein scan converter 46 processes the echo signals for display in the desired image format on display unit 48. Graphics generator 56 also provides images for being displayed on display unit 48, as discussed further herein with respect to the various embodiments.

For real-time volumetric imaging, the ultrasound diagnostic imaging system includes a 3D image rendering processor which receives image lines from the signal processor 44 for the rendering of a real-time three dimensional image which can be displayed on the display unit 48. The ultrasound system display unit 48 can be used to view cardiac images during an acquisition of the same. The cardiac images may include sector-shaped images, such as four-chamber views of the heart. A sequence of real-time images can be acquired by placement of the probe for an apical 4-chamber view of the heart, in which the probe is oriented to view the heart from the proximity of the heart's apex. The largest chamber in the four-chamber view of the heart, generally observed in the central and upper right portion of the image, is the left ventricle (LV).

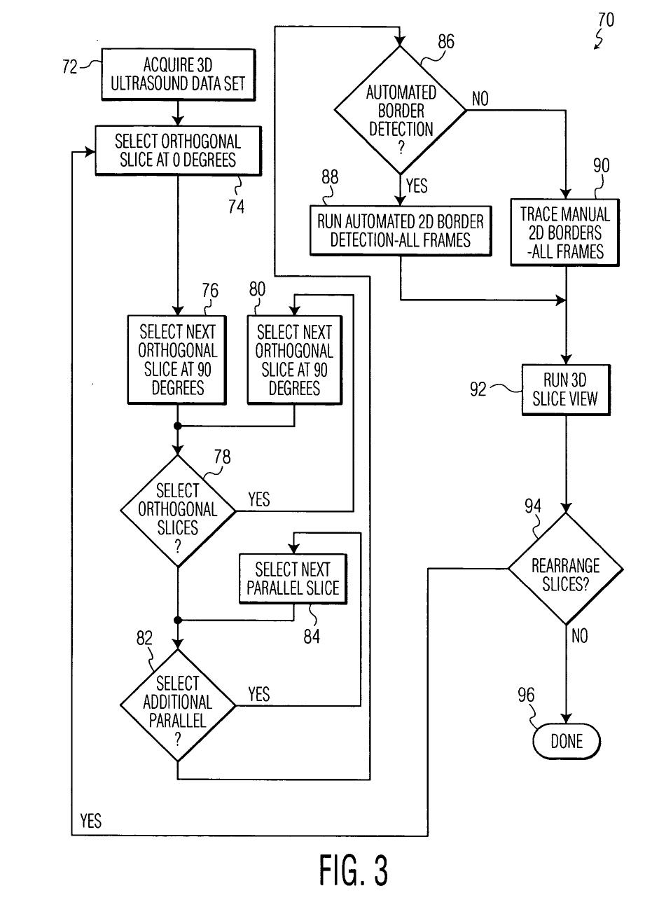

FIG. 3 is a flow diagram view of a method for generating a three-dimensional (3D) qualitative display of 2D LV border tracings in an ultrasound diagnostic imaging system according to one embodiment of the present disclosure. In a first step 72, a 3D ultrasound data set of an image, for example, a heart, is acquired using suitable techniques known in the art. In step 74, a first orthogonal 2D slice is selected at zero (0) degrees. The process proceeds with selection of a next orthogonal slice at ninety (90) degrees, as indicated by reference numeral 76. In other words, the first 2D slice is orthogonal at zero degrees to the second 2D slice at 90 degrees from the first 2D slice.

At step 78, a query is conducted whether to select another orthogonal 2D slice. If yes, then the process proceeds to step 80 for the selection of a next orthogonal slice at ninety (90) degrees. The process then repeats with the query at step 78. In response to non-selection of another orthogonal slice at step 78, the process proceeds to step 82. Step 82 queries whether any parallel 2D slices are desired. Parallel 2D slices are defined herein as being orthogonal to both the first 2D slice and the second 2D slice. If a parallel 2D slice is desired, then the process proceeds to step 84 for the selection of a parallel 2D slice. The process then repeats with the query at step 82.

Subsequent to no further selection of parallel 2D slices, the process proceeds to step 86. Step 86 includes a query whether automated border detection is desired. If automated border detection is desired, then the process proceeds to step 88. In step 88, an automated 2D border detection routine is run for all frames of a sequence. The sequence includes at least two or more of the orthogonal 2D slice at zero degrees, the orthogonal 2D slice at 90 degrees, and any parallel 2D slices.

In query 86, if automated border detection is not desired, then the process proceeds to step 90. In step 90, a manual 2D border detection routine is run for all frames of the sequence. As mentioned above, the sequence includes at least two or more of the orthogonal 2D slice at zero degrees, the orthogonal 2D slice at 90 degrees, and any parallel 2D slices. Subsequent to either of step 88 or 90, the process then proceeds to step 92.

In step 92, a 3D slice view is run, as will be discussed further herein below. The 3D slice view includes a display view of border tracings of the orthogonal slices along a first axis and border tracings of the parallel slices along a second axis orthogonal to the first axis. An example illustration of a 3D slice view is shown in FIG. 1.

Subsequent to the running of the 3D slice view in step 92, the method includes a query at step 94. The query at step 94 asks whether to rearrange the 2D slices of the 3D slice view. If rearranging slices is selected, then the process returns to step 74 and proceeds as discussed. If no rearranging of slices is selected, then the process ends at step 96.

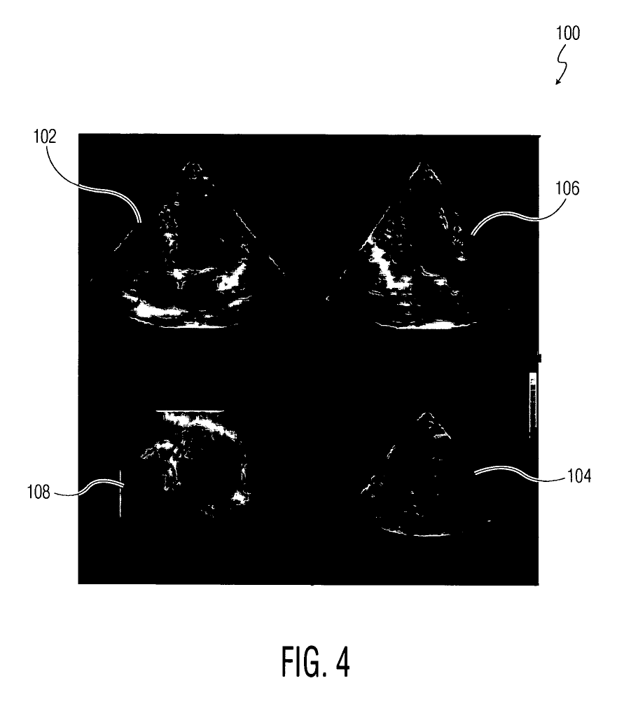

FIG. 4 is an illustrative view 100 of a 3D volume and portions thereof according to one embodiment of the present disclosure. The upper left corner of FIG. 4 denoted by reference numeral 102 illustrates a first 2D slice of a 3D volume of ultrasound data. For example, the first 2D slice can include a slice obtained from the 3D volume data set, wherein a display of the 3D volume view is shown in the lower right corner of FIG. 4, indicated by reference numeral 104. The upper right corner of FIG. 4, denoted by reference numeral 106, illustrates a second 2D slice of the same 3D volume of ultrasound data. The second 2D slice can include a slice that is also obtained from the 3D volume data set, wherein the display of the 3D volume view 104 is shown in the lower right corner of FIG. 4. The second 2D slice is selected so as to be orthogonal at 90 degrees to the plane of the first 2D slice at zero (0) degrees.

The lower left corner of FIG. 4 is denoted by reference numeral 108 and illustrates an example of a parallel 2D slice of the 3D volume of ultrasound data. That is, the parallel 2D slice includes a slice obtained from the 3D volume data set, such as that illustrated by the 3D volume view 104 in the lower right corner of FIG. 4. The parallel 2D slice is selected to be orthogonal to the plane of the first 2D slice and orthogonal to the plane of the second 2D slice. The 3D volume of ultrasound data and the 2D slices selected there from can be obtained using any suitable techniques known in the art.

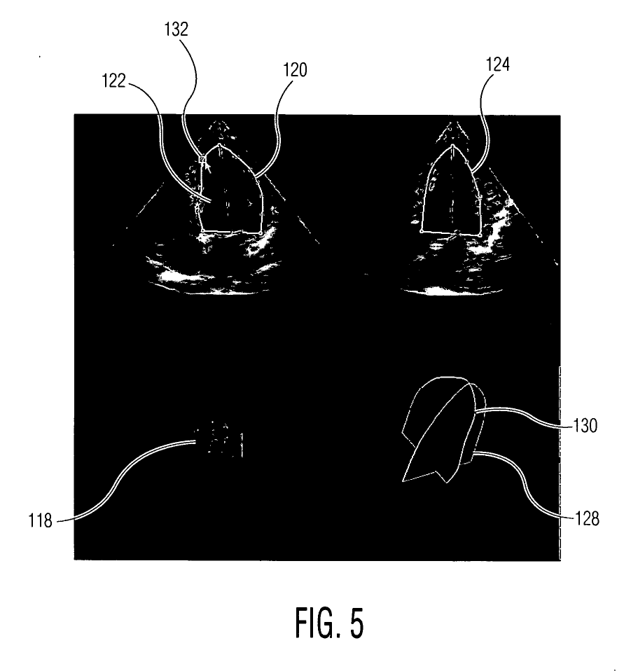

In each of FIGS. 5, 6, 7, and 8, in a display view 110, there is shown a heart blood pool corresponding to the dark portion of the respective images, as indicated by the reference numeral 120. In the upper left corner slice (corresponding to a first vertical 2D slice of a 3D volume), a manual border trace is shown as indicated by reference numeral 122. Manual border tracing can be accomplished using input from a system operator or clinician to define a ROI. Alternatively, the border tracing could be accomplished using automated border tracing techniques, such as disclosed in U.S. Patent Application Ser. No. 60/507,263, filed Sep. 29, 2003, entitled “Ultrasonic Cardiac Volume Quantification,” assigned to the assignee of the present application (Attorney docket number US030379) and incorporated herein by reference.

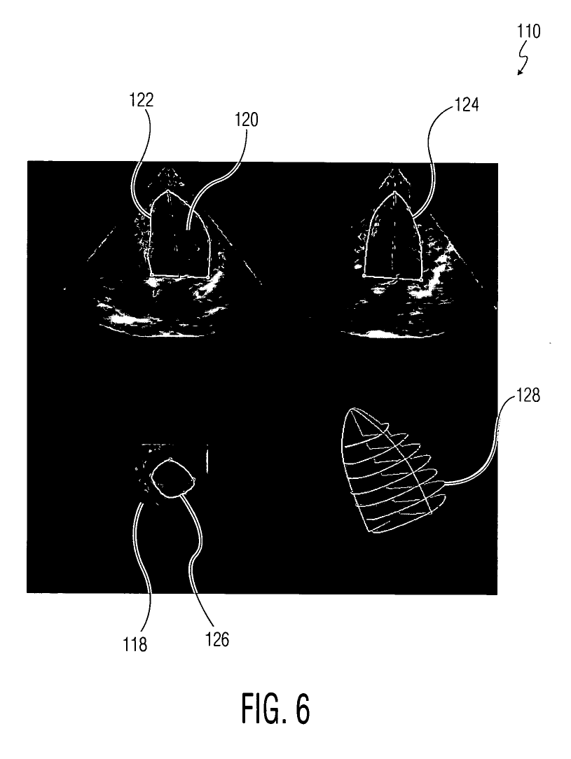

In the upper right corner slice (corresponding to a second vertical 2D slice of the 3D volume, the second vertical 2D slice being orthogonal to the first vertical 2D slice), a manual border trace on the second 2D slice is shown as indicated by reference numeral 124. In the lower left corner, a parallel 2D slice 118 of the 3D volume orthogonal to the vertical 2D slices of the upper left and right corners is shown. A border trace can be performed on the parallel 2D slice 118 of the lower left corner as shown in FIGS. 6, 7, and 8 and further as indicated by reference numeral 126. In addition, additional border traces of the lower left corner can be obtained from additional parallel 2D slices (not shown) that are orthogonal to the 2D slices of the upper left and right corners, similar to that of the lower left corner of FIGS. 6, 7, and 8.

The example shown in FIG. 1 illustrates border traces of seven (7) parallel 2D slices, as indicated by reference numerals 12-24, of a 3D volume orthogonal to the vertical 2D slices, as indicated by reference numerals 26 and 28. In one embodiment, the multiple border traces obtained from parallel 2D slices orthogonal to the vertical 2D slices of the upper left and right corners of FIGS. 6-8 may include up to nine (9) parallel 2D slices.

Furthermore, in the lower right corner 128 of the display view 110 in each of FIGS. 5-8, according to one embodiment of the present disclosure, the method includes rendering a composite display that shows a combination of the vertical axis and horizontal axis border traces. Ideally, all vertical axis border traces should line up with respect to a common horizontal axis. Similarly, all horizontal axis border traces should line up with respect to a common vertical axis. With proper horizontal and vertical axis alignments, the shape of the blood pool in 3D can be substantially accurately ascertained.

On the other hand, if the horizontal and vertical axis alignments are not aligned (i.e., misaligned), then the misalignments provide information at least sufficient to indicate that an appropriate corrective measure (or measures) is needed to be taken. In other words, the misalignment, as may appear in the composite display (for example, as shown in FIGS. 1 and 10), provides an indication of where one or more problem may exist. Alternatively, the misalignment can be indicative that the 3D data set is in error and that there is a need to rearrange the MPR slices or to repeat the data acquisition for the particular volume or region of interest.

In FIG. 5, there are two long axis border traces, 122 and 124. In the lower right corner of FIG. 5, the display 110 includes a dot cursor 130. The dot cursor 130 has been provided for corresponding with a boxed dot cursor 132 of the upper left corner of FIG. 5 in three dimensional space. With a color display, dot cursor 130 may include a red dot cursor, whereas boxed dot cursor 132 may include a green dot cursor. FIG. 6 contains nine (9) border traces, corresponding to two (2) long axis border traces and seven (7) short axis border traces.

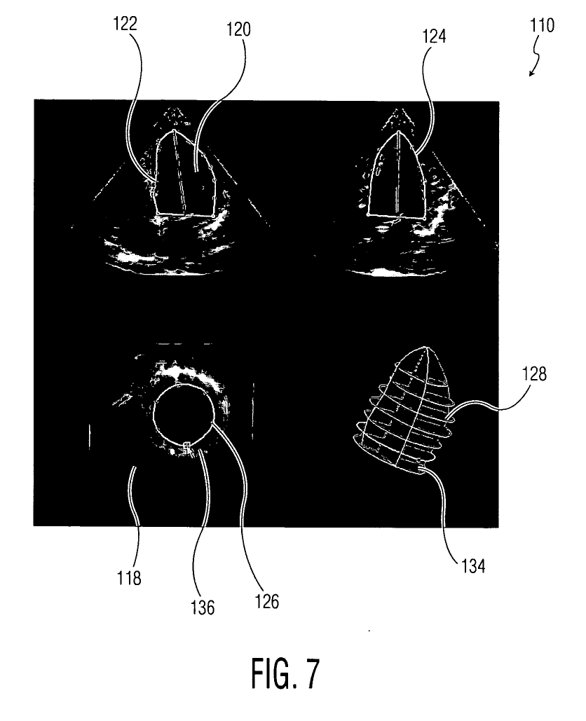

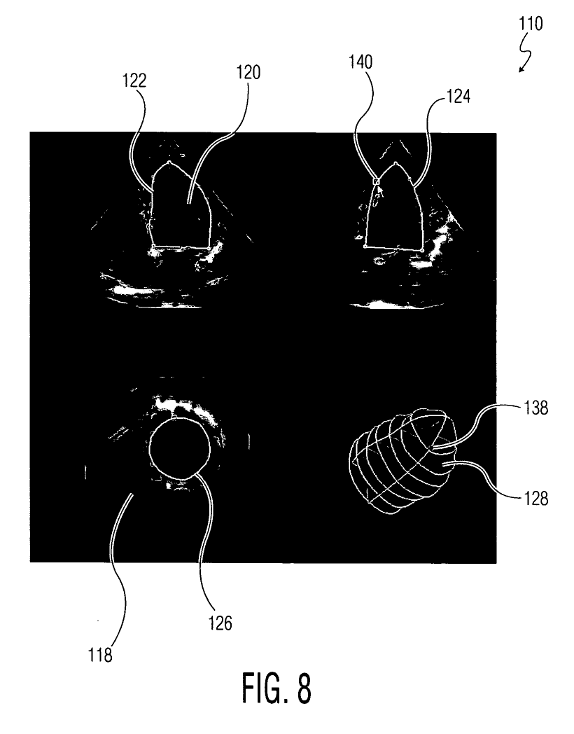

FIG. 7 contains nine (9) traces, corresponding to two (2) long axis border traces and seven (7) short axis border traces. In the lower right corner of FIG. 7, a dot cursor indicated by reference numeral 134 corresponds to a boxed dot cursor 136 in the lower left corner of FIG. 7 in three dimensional space. With a color display, dot cursor 134 may include a red dot cursor, whereas boxed dot cursor 136 may include a green dot cursor. FIG. 8 is the same as FIG. 7, but with the 3D object 128 at a different angle. In the lower right corner of FIG. 8, the dot cursor 138 corresponds to the boxed dot cursor 140 of the upper right corner of FIG. 8 in three dimensional space.

With reference to the display view 110 of FIGS. 5, 7, and 8, the ultrasound system is configured for providing at least one reference point on a border tracing within a 2D slice view. The at least one reference point of the 2D slice view corresponds to a like reference point in the 3D view generated by the 3D data set. Providing the reference point enables a clinician to more readily understand where in 3 dimensional space a given point is located within the various views. The reference point can be incorporated into respective drawing views as a function of 3D data set and using data processing techniques known in the art.

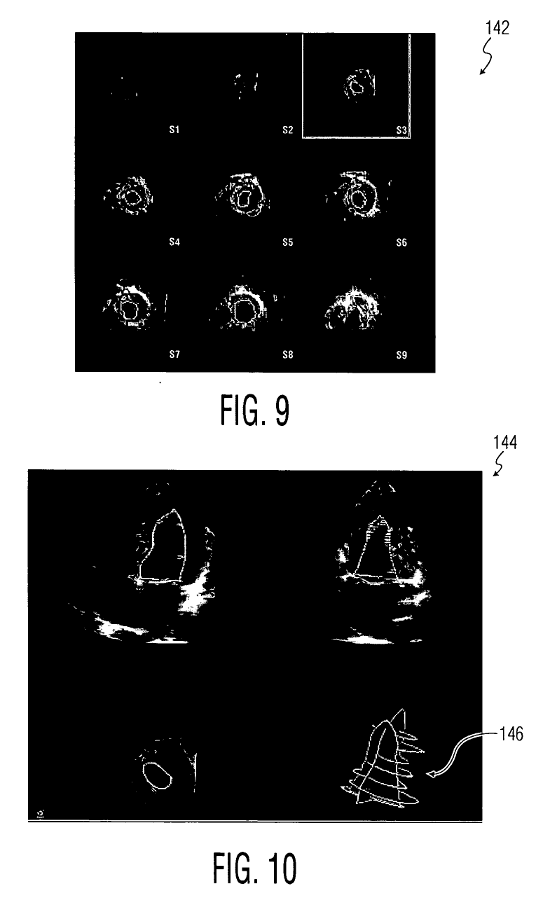

FIG. 9 is an illustrative view of various parallel 2D slices of a 3D volume according to one embodiment of the present disclosure. More particularly, the display view 142 of parallel 2D slices S1-S9 are representative of the parallel 2D slices shown in FIGS. 5-8. In addition, FIG. 10 is an illustrative view 144 of a 3D volume and portions thereof, including a three-dimensional (3D) display view 146 of short axis border tracings and long axis border tracings of the target volume according to one embodiment of the present disclosure, similarly as discussed with respect to FIGS. 5-8.

On advantage of the present embodiments is that they provide clinical usefulness. For example, the embodiments provide a display that shows how a series of 2D borders drawn manually or automatically on MPR views line up in 3D space. If the apex of the heart is selected incorrectly, such as by a selection of an incorrect MPR slice, then the error will show up as a misalignment of the 2D borders. FIG. 1, as discussed herein is an example of such a misalignment. Accordingly, in response to viewing the display of the 2D border misalignment, a clinician or physician would know that corrective action would be needed. Such corrective action could include either selecting new MPR slices or to redo the 2D borders, depending upon the type of misalignment.

In addition, the embodiments of the present disclosure also include the provision of a “dot cursor” 138 in the 3D space view, for example, as illustrated on the lower right portion of FIG. 8. During display of the 2D and 3D images, positioning of a mouse pointer on any of the three 2D image views, whether the upper left, upper right, or lower left images, causes a corresponding movement of the dot cursor 138 in the 3D view. For example, dot cursor 138 indicates in 3D space the location of where the mouse pointer is currently pointing to in the 2D space, as indicated by the dot cursor 140. In a color display, dot cursor 138 can include a red dot cursor and dot cursor 140 can include a green dot cursor.

Accordingly, a system user or clinician can use the dot cursor to assist in visualizing where the object being pointed to in a 2D image is in 3D space. This is particularly helpful when the clinician is performing manual tracing of 2D borders and checking for alignment. Responsive to the mouse pointer pointing to a green dot cursor which a user had placed for a manual border trace, the green dot cursor is highlighted with a box positioned around the corresponding green dot cursor. In addition, the red dot cursor moves or maps to the corresponding location in 3D space. In other words, the dot cursor 140 points to a position on a border of 2D MPR slice (which could be selected from any of the three views, i.e., upper left, upper right, and lower left, as needed for a given diagnostic analysis) and in response thereto, a corresponding dot cursor 138 is provided on the lower right view in 3D space. Accordingly, this provides a clinician or physician with an interactive tool, to move around in 2D space in the different views and see where a selected given point is located in the 3D volume view.

In one embodiment, ultrasound diagnostic imaging system 30 includes computer software configured, using programming techniques known in the art, for carrying out the various functions and functionalities as described herein. Responsive to an input selection of a location within one of the 2D slice views using a pointer device (such as a computer mouse or other input device), the program provides a dot cursor within the 3D view of the border tracings. As discussed herein above with respect to dot cursor 138 and dot cursor 140, positioning of a first dot cursor 140 may be placed in response to interactive user input, and wherein responsive to positioning of the first dot cursor 140, the ultrasound diagnostic imaging system places the second dot cursor 138 within the 3D view.

Alternatively, positioning of the first dot cursor 140 may be placed automatically by the ultrasound imaging system, such as to a default location, and responsive to positioning of the first dot cursor, the ultrasound imaging system places the second dot cursor 138, wherein the second dot cursor indicates a location in 3D space with the second dot cursor corresponding to the location where the first dot cursor is. In other words, the ultrasound imaging system further includes displaying a first dot cursor positioned within a 2D view of the display, wherein responsive to displaying of the first dot cursor, the system is further configured for displaying a second dot cursor in the composite 3D view of the border tracings, wherein the second dot cursor indicates a location in 3D space corresponding to location of the first dot cursor in a 2D slice.

According to one embodiment of the present disclosure, a method for generating a three-dimensional (3D) qualitative display in an ultrasound system includes generating first. and second two-dimensional (2D) slices from a 3D data set. For example, the 3D can include a data set used to generate a 3D volume view of an ultrasound image. The first slice defines a first plane of the 3D volume view along a first axis. The second 2D slice defines a second plane of the 3D volume view along the first axis, wherein the second plane is orthogonal to the first plane. Subsequent to generating the first and second 2D slices, the method includes generating first and second border tracings around a region or portion of interest in the first and second 2D slices, respectively. Moreover, the first and second border tracings can include manual border tracings and/or automatic border tracings.

The method also includes displaying representations of the first and second border tracings within a single 3D view. Displaying representations of the first and second border tracings facilitates a 3D view that provides an indication of alignment distortion of the first and second border tracings along the first axis. The displaying of the 3D view of representations of the first and second border tracings can also include separately displaying at least an image of the first 2D slice and the second 2D slice within a single display view.

The method according to another embodiment of the present disclosure further includes the generating of a third 2D slice from the 3D data set of the 3D volume view. The third 2D slice defines a third plane of the 3D volume view, wherein the third plane is orthogonal to the first and second planes. In addition, the method includes generating a third border tracing around the portion of interest in the third slice. Furthermore, displaying can also include displaying a representation of the third border tracing. Accordingly, the 3D view further provides an indication of alignment distortion of the first, second, and third border tracings along the first and second axes. In addition, displaying the 3D view of the first, second, and the third border tracing representations can also include separately displaying at least an image of the first 2D slice, the second 2D slice, and the third 2D slice within a single display view.

In one embodiment, the first axis corresponds to a long axis and the second axis corresponds to a short axis. In addition, the first, second and third border tracings can include manual border tracings and/or automatic border tracings.

In addition, in yet another embodiment of the present disclosure, the generating of the third 2D slice includes generating at least one additional 2D slice parallel to the third 2D slice. The at least one additional 2D slice defines at least one additional plane of the 3D volume view. Furthermore, the method includes generating at least one additional border tracing around the region or portion of interest in the at least one additional 2D slice. In one embodiment, generating the at least one additional 2D slice includes generating up to nine additional parallel 2D slices.

In addition, displaying also includes displaying a representation of the at least one additional border tracing, wherein the 3D view further provides an indication of alignment distortion of the at least one additional border tracing along the first and second axes. Displaying the 3D view of the first, second, third border and the at least one additional border tracing representations can further include separately displaying one or more image of the first 2D slice, the second 2D slice, the third 2D slice, and the at least one additional 2D slice.

In yet another embodiment, a method for generating a three-dimensional (3D) qualitative display of border tracings of a 3D volume view derived from a 3D data set of an ultrasound image in a region of interest obtained using an ultrasound diagnostic imaging system includes the following. A first two-dimensional (2D) slice is generated from the 3D ultrasound image data set used to generate a 3D volume view of an ultrasound image in a region of interest. The first 2D slice defines a first plane of the 3D volume view along a first axis. Next, a second 2D slice is generated from the 3D data set of the 3D volume view, the second 2D slice defining a second plane of the 3D volume view along the first axis. In one embodiment, the second plane is selected to be orthogonal to the first plane.

Subsequent to generating the first and second slices, at least one additional 2D slice is generated from the 3D data set of the 3D volume view. The at least one additional 2D slice defines at least one additional plane of the 3D volume view along a second axis. In one embodiment, the at least one additional plane is orthogonal to the first and second planes. The process continues with generating first, second, and at least one additional border tracing around a region or portion of interest in the first 2D slice, the second 2D slice, and the at least one additional 2D slice, respectively.

A 3D view of the first and second border tracings along the first axis and the at least one additional border tracing along the second axis are then displayed within a display view. The 3D view advantageously provides an indication of alignment distortion of the first, second, and at least one additional border tracing along the first and second axes. Displaying within the display view can further include separately displaying one or more of the following: of n image of the first 2D slice, an image of the second 2D slice, and an image of the at least one additional 2D slice.

According to another embodiment of the present disclosure, an ultrasound diagnostic imaging system includes at least a processor and a display, the ultrasound diagnostic imaging system for performing the method of generating a three-dimensional (3D) qualitative display as discussed herein. In particular, responsive to instructions stored on a computer readable storage medium and executable by the processor, the processor generates a first two-dimensional (2D) slice of a 3D data set that is used to generate a 3D volume view of an ultrasound image. The first slice defines a first plane of the 3D volume view along a first axis. The processor further generates a second 2D slice from the 3D data set, the second slice defining a second plane of the 3D volume view along the first axis. The second plane is orthogonal to the first plane. The processor is further adapted to generate a first and a second border tracing around a portion of interest in the first and second slices, respectively. Border tracing can be accomplished via manual or automatic border tracing, as discussed herein. Furthermore, the processor couples to the display, wherein the display is configured to display representations of the first and second border tracings within a single 3D view. The 3D view provides an indication of alignment distortion of the first and second border tracings along the first axis.

In another embodiment, the processor of the ultrasound diagnostic system is further responsive to computer readable instructions for generating a third 2D slice from the 3D data set of the 3D volume view. The third slice defines a third plane of the 3D volume view, wherein the third plane is orthogonal to the first and second planes. In addition, the processor is adapted to further generate a third border tracing around the region of interest in the third slice. The display is further for displaying a representation of the third border tracing within the single 3D view, wherein the 3D view further provides an indication of alignment distortion of the first, second, and third border tracings along the first and second axes.

In yet another embodiment, the processor is further for generating at least one additional 2D slice, the at least one additional 2D slice defining at least one additional plane of the 3D volume view along the second axis. The at least one additional plane is orthogonal to the first and second planes. In addition, the processor is for generating at least one additional border tracing around the region of interest in the at least one additional 2D slice. Furthermore, the display is further for displaying a representation of the at least one additional border tracing within the single 3D view, wherein the 3D view further provides an indication of alignment distortion of the at least one additional border tracing along the first axis and second axes.

The ultrasound diagnostic imaging system is further configured for implementing the method according to the various embodiments of the present disclosure as discussed herein. Programming of the computer readable instructions for implementation of the method of the various embodiments of the present disclosure by the processor can be performed using programming techniques known in the art.

The embodiments of the present disclosure have been described in connection with acquisition of image data using a digital beamformer. It will be understood that the embodiments may be applied to analog implementations of ultrasound imaging systems.

Although only a few exemplary embodiments have been described in detail above, those skilled in the art will readily appreciate that many modifications are possible in the exemplary embodiments without materially departing from the novel teachings and advantages of the embodiments of the present disclosure. Accordingly, all such modifications are intended to be included within the scope of the embodiments of the present disclosure as defined in the following claims. In the claims, means-plus-function clauses are intended to cover the structures described herein as performing the recited function and not only structural equivalents, but also equivalent structures.

1. A method for generating a three-dimensional (3D) qualitative display in an ultrasound system comprising:
generating a first two-dimensional (2D) slice from a 3D data set that is used to generate a 3D volume view of an ultrasound image, the first slice defining a first plane of the 3D volume view along a first axis;
generating a second 2D slice from the 3D data set of the 3D volume view, the second slice defining a second plane of the 3D volume view along the first axis, wherein the second plane is orthogonal to the first plane;
generating a first and a second border tracing around a portion of interest in the first and second 2D slices, respectively; and
displaying representations of the first and second border tracings within a single 3D view, wherein the 3D view provides an indication of alignment distortion of the first and second border tracings along the first axis.

2. The method of claim 1, further comprising:
generating a third 2D slice from the 3D data set of the 3D volume view, the third slice defining a third plane of the 3D volume view, wherein the third plane is orthogonal to the first and second planes; and
generating a third border tracing around the portion of interest in the third slice, wherein displaying also includes displaying a representation of the third border tracing, and wherein the 3D view further provides an indication of alignment distortion of the first, second, and third border tracings along the first and second axes.

3. The method of claim 2, wherein generating the third 2D slice includes generating at least one additional 2D slice parallel to the third 2D slice, the at least one additional 2D slice defining at least one additional plane of the 3D volume view; and generating at least one additional border tracing around the portion of interest in the at least one additional 2D slice, wherein displaying also includes displaying a representation of the at least one additional border tracing, and wherein the 3D view further provides an indication of alignment distortion of the at least one additional border tracing along the first and second axes.

4. The method of claim 2, wherein the first axis corresponds to a long axis and the second axis corresponds to a short axis.

5. The method of claim 1, wherein the first and second border tracings include at least one selected from the group consisting of manual border tracings and automatic border tracings.

6. The method of claim 2, wherein the first, second and third border tracings include at least one selected from the group consisting of manual border tracings and automatic border tracings.

7. The method of claim 3, wherein generating the at least one additional 2D slice includes up to nine additional parallel 2D slices.

8. The method of claim 1, wherein the displaying of the 3D view of representations of the first and second border tracings further includes separately displaying at least an image of the first 2D slice and the second 2D slice within a single display view.

9. The method of claim 2, wherein displaying the 3D view of the representations of the first, second, and third border tracings further includes separately displaying at least an image of the first 2D slice, the second 2D slice, and the third 2D slice within a single display view.

10. The method of claim 3, wherein displaying the 3D view of the representations of the first, second, third border tracings and the at least one additional border tracing further includes separately displaying at least an image of the first 2D slice, an image of the second 2D slice, and an image of at least one selected from the group consisting of the third 2D slice and the at least one additional 2D slice.

11. A method for generating a three-dimensional (3D) qualitative display of border tracings of a 3D volume view derived from a 3D data set of an ultrasound image in a region of interest obtained using an ultrasound diagnostic imaging system, the method comprising:
generating a first two-dimensional (2D) slice from the 3D ultrasound image data set used to generate a 3D volume view of an ultrasound image in a region of interest, the first 2D slice defining a first plane of the 3D volume view along a first axis;
generating a second 2D slice from the 3D data set of the 3D volume view, the second 2D slice defining a second plane of the 3D volume view along the first axis, wherein the second plane is orthogonal to the first plane;
generating at least one additional 2D slice from the 3D data set of the 3D volume view, the at least one additional 2D slice defining at least one additional plane of the 3D volume view along a second axis, wherein the at least one additional plane is orthogonal to the first and second planes;
generating a first, a second, and at least one additional border tracing around a portion of interest in the first 2D slice, the second 2D slice, and the at least one additional 2D slice, respectively; and
displaying a 3D view of the first and second border tracings along the first axis and the at least one additional border tracing along the second axis within a display view, the 3D view providing an indication of alignment distortion of the first, second, and at least one additional border tracing along the first and second axes.

12. The method of claim 1 1, wherein displaying within the display view further includes separately displaying at least one selected from the group consisting of an image of the first 2D slice, an image of the second 2D slice, and an image of the at least one additional 2D slice.

13. An ultrasound diagnostic system comprising:
a processor for: generating a first two-dimensional (2D) slice of a 3D data set that is used to generate a 3D volume view of an ultrasound image, the first slice defining a first plane of the 3D volume view along a first axis;
generating a second 2D slice from the 3D data set that is used to generate the 3D volume view, the second slice defining a second plane of the 3D volume view along the first axis, wherein the second plane is orthogonal to the first plane;
generating a first and a second border tracing around a portion of interest in the first and second slices, respectively; and
a display for displaying representations of the first and second border tracings within a single 3D view, wherein the 3D view provides an indication of alignment distortion of the first and second border tracings along the first axis.

14. The ultrasound diagnostic system of claim 13, wherein said processor is further for: generating a third 2D slice from the 3D data set of the 3D volume view, the third slice defining a third plane of the 3D volume view, wherein the third plane is orthogonal to the first and second planes; and
generating a third border tracing around the portion of interest in the third slice, and wherein said display is further for displaying a representation of the third border tracing within the single 3D view, wherein the 3D view further provides an indication of alignment distortion of the first, second, and third border tracings along the first and second axes.

15. The ultrasound diagnostic system of claim 14, wherein said processor is further for:
generating at least one additional 2D slice, the at least one additional 2D slice defining at least one additional plane of the 3D volume view along the second axis, wherein the at least one additional plane is orthogonal to the first and second planes; and
generating at least one additional border tracing around the portion of interest in the at least one additional 2D slice, and wherein said display is further for displaying a representation of the at least one additional border tracing within the single 3D view, wherein the 3D view further provides an indication of alignment distortion of the at least one additional border tracing along the first axis and second axes.

16. The ultrasound diagnostic system of claim 14, wherein the first axis corresponds to a long axis and the second axis corresponds to a short axis.

17. The ultrasound diagnostic system of claim 13, wherein the border tracing includes one selected from the group consisting of manual border tracing and automatic border tracing.

18. The ultrasound diagnostic system of claim 14, wherein the border tracing includes one selected from the group consisting of manual border tracing and automatic border tracing.

19. The ultrasound diagnostic system of claim 15, wherein generating at least one additional slice includes up to nine additional parallel 2D slices.

20. The ultrasound diagnostic system of claim 13, wherein said display for displaying the 3D view of representations of the first and second border tracings is further for separately displaying at least an image of the first 2D slice and the second 2D slice within a single display view.

21. The ultrasound diagnostic system of claim 14, wherein said display for displaying the 3D view of representations of the first, second, and third border tracings is further for separately displaying at least an image of the first 2D slice, the second 2D slice, and the third 2D slice within a single display view.

22. The ultrasound diagnostic system of claim 15, wherein said display for displaying the 3D view of representations of the first, second, third border, and at least one additional border tracings is further for separately displaying at least an image of the first 2D slice, an image of the second 2D slice, and an image of at least one selected from the group consisting of the third and the at least one additional slice within a single display view.

23. An ultrasound diagnostic system for generating a 3D qualitative display of border tracings of a 3D volume view derived from a 3D data set of an ultrasound image in a region of interest, the system comprising:
a processor for:
generating a first two-dimensional (2D) slice of a 3D ultrasound image data set used to generate a 3D volume view of an ultrasound image in a region of interest, the first 2D slice defining a first plane of the 3D volume view along a first axis;
generating a second 2D slice from the 3D data set of the 3D volume view, the second slice defining a second plane of the 3D volume view along the first axis, wherein the second plane is orthogonal to the first plane;
generating at least one additional 2D slice from the 3D data set of the 3D volume view, the at least one additional 2D slice defining at least one additional plane of the 3D volume view along a second axis, wherein the at least one additional plane is orthogonal to the first and second planes;
generating a first, a second, and at least one additional border tracing around a portion of interest in the first 2D slice, the second 2D slice, and the at least one additional 2D slice, respectively; and
means for displaying a 3D view of the first and second border tracings along the first axis and displaying the at least one additional border tracing along the second axis within a display view, the 3D view providing an indication of alignment distortion of the first, second, and at least one additional border tracing along the first and second axes.

24. The ultrasound diagnostic system of claim 23, wherein said means for displaying is further for separately displaying at least one selected from the group consisting of an image of the first 2D slice, an image of the second 2D slice, and an image of the at least one additional 2D slice on the single display view.

25. The ultrasound diagnostic system of claim 14, wherein the ultrasound imaging system further includes displaying a first dot cursor positioned within a 2D view of the display, wherein responsive to displaying the first dot cursor, the system is further configured for displaying a second dot cursor in a representative position of the 3D view of the border tracings, wherein the second dot cursor indicates a location in 3D space corresponding to the location of the first dot cursor in the 2D view.

26. The method of claim 2, further comprising: displaying a first dot cursor positioned within a 2D view of the display, wherein responsive to displaying of the first dot cursor, the system is further configured for displaying a second dot cursor in the 3D view of the border tracings, wherein the second dot cursor indicates a location in 3D space on a 2D slice of the location of the first dot cursor.
